(19) United States
(12) Patent Application Publication
(10) Pub. No.: US 2012/0016018 A1
(43) Pub. Date: Jan. 19, 2012

(54) COMPOSITION FOR INDUCING MULTIPLE NUCLEAR DIVISION OF CELLS

(76) Inventor: Gee-Hwoon LEE, Seoul (KR)

(21) Appl. No.: 12/846,322

(22) Filed: Jul. 29, 2010

(30) Foreign Application Priority Data
Jul. 13, 2010 (KR) 10-2010-0067622

(51) Int. Cl. A61K 31/351; A61P 37/00; A61P 9/00; C07D 309/32; C12N 5/00

(52) U.S. Cl. 514/459; 549/419; 435/375

(57) ABSTRACT

The present invention relates to the novel use of a composition comprising a compound of the formula I:

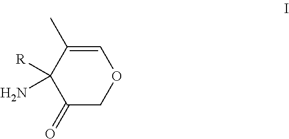

wherein R is C₂H₅ or C₂H₃,
or a pharmaceutically acceptable salt thereof, for inducing multiple nuclear divisions of cells; inducing tissues, blood vessels or organs to regenerate after the multiple nuclear divisions; or inducing stem cells to differentiate into immunocytes after the multiple nuclear divisions.

### CROSS-REFERENCE TO RELATED APPLICATIONS

This application claims the benefit and priority of Korean Application No. 10-2010-0067622, filed Jul. 13, 2010. The entire disclosure of the above application is incorporated herein by reference.

### TECHNICAL FIELD

The present invention relates to the novel use of a composition comprising a compound of the formula I or a pharmaceutically acceptable salt thereof. Specifically, the composition according to the invention can induce multiple nuclear divisions of cells, promote the regeneration of blood vessels or tissues, and induce stem cell differentiation into immunocytes. The composition according to the invention can also treat or prevent diseases such as cardiovascular diseases and immune deficiency.

### BACKGROUND ART

Stem cells are characterized by the ability to renew themselves through mitotic cell division and differentiate into a diverse range of specialized cell types. They can be divided into totipotent stem cells, pluripotent stem cells, multipotent stem cells, etc.

Many different studies have been conducted on the method for regenerating injured or damaged tissues or organs using stem cells. However, it is not yet known which composition comprising a specific compound can regenerate injured blood vessels, tissues or organs with efficiency in a short time, and how cells required to regenerate are supplemented.

### DISCLOSURE

### Technical Problem

It is the object of the present invention to provide a composition to induce multiple nuclear divisions of cells, thereby regenerate blood vessels, tissues or organs.

It is another object of the present invention to provide a composition to induce stem cells to differentiate into immunocytes.

It is another object of the present invention to provide a composition for treating or preventing diseases associated with cardiovascular function or immune deficiency.

### Technical Solution

The present invention relates to the novel use of a composition comprising a compound of the formula I:

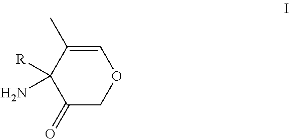

wherein R is C₂H₅ or C₂H₃,

or a pharmaceutically acceptable salt thereof, for inducing multiple nuclear divisions of cells.

The invention also relates to the novel use of the composition for inducing tissues, blood vessels or organs to regenerate after the multiple nuclear divisions.

The invention also relates to the novel use of the composition for inducing stem cells to differentiate into immunocytes after the multiple nuclear divisions.

The composition comprising the compound of the formula I or a pharmaceutically acceptable salt thereof according to the invention can comprise pharmaceutically acceptable carriers. And, the composition can additionally comprise other ingredients as long as the activity of the compound of the formula I or a pharmaceutically acceptable salt thereof is not reduced. The pharmaceutically acceptable salts or carriers are well known in the art, and can be selected by person having ordinary skill in the art.

The dose and duration of the treatment will depend on a variety of factors, including the age, body weight, general health, sex, diet and the type of disease of the patient. The composition according to the invention can be administered by all types of route available in the art. For example, the composition according to the invention can be administered by parenteral (e.g. subcutaneously, intramuscularly, intravenously, intraperitoneally, intrapleurally, intravesicularly or intrathecally), topical, oral, rectal, nasal route, etc.

The composition according to the invention stimulates adult stem cells in bone marrow. The stimulated adult stem cells generate multinuclear genesis cells. And then, about 80% of genesis cells immediately differentiate into immunocytes, such as NK cells (22.3˜30%), T cells (32%), B cells (16˜23.7%), thrombocytes (2%), etc. that perform the immune functions.

It can be confirmed from experiments using nude mice that the composition according to the invention differentiates stem cells into immunocytes. The nude mice are athymic caused by a developmental failure of the thymic anlage. Consequently, homozygous nude mice lack T cells and suffer from a lack of cell-mediated immunity. Because of a defect in helper T-cell activity, there are no responses to thymus-dependent antigens.

However, when the composition according to the invention is administered to nude mice, all of the cells, tissues and organs, which have receptors to be able to receive signals, undergo processes such as differentiation, proliferation or recovery of cells. As a result, T cells are observed in blood of nude mice by the composition according to the invention.

In this case, T cells observed are generated by two routes.

One route is that T cells precursors circulating in the blood, which did not differentiate or proliferate by a developmental failure of the thymic anlage, receive the signals derived from the composition of the invention, and differentiate into T cells.

And, another route is that the composition according to the invention directly stimulates adult stem cells in bone marrow, and then the generated NK and NKT cells redifferentiate into T cells, B cells, etc.

The nude mice model experiments showing that the composition according to the invention differentiates stem cells into immunocytes are provided in the following Examples.

### Advantageous Effects

The composition comprising a compound of the formula I or a pharmaceutically acceptable salt thereof according to the invention can induce multiple nuclear divisions of cells, thereby regenerate blood vessels, tissues or organs. Furthermore, the composition according to the invention can induce stem cells to differentiate into immunocytes, thereby increase the level of immunity in patients with weakened immune systems or patients suffered from immune deficiency, and treat or prevent diseases associated with cardiovascular function or immune deficiency.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the ¹H NMR spectrum of the compound of the formula II.

FIG. 2 shows the ¹H NMR spectrum of the compound of the formula III.

FIG. 3 is the photomicrograph showing multiple nuclear divisions occurring in the cell after HME 50 HT cells were treated with the composition according to the invention.

FIG. 4 is the magnified image of FIG. 3.

FIGS. 5A-D are photographs taken at 24 hours after treatment of HME cells with the composition according to the invention or without any agents. The top photographs show the experimental group, whereas the bottom photographs show the untreated control group.

FIGS. 6A-F are photographs taken at 72 hours after treatment of HME cells with the composition according to the invention (the experimental group).

FIGS. 7A-D are photographs taken at 72 hours after treatment of HME cells without any agents (the untreated control group).

FIGS. 8A-D are photographs taken at 24 hours after treatment of HMS cells with the composition according to the invention or without any agents. The top photographs show the experimental group, whereas the bottom photographs show the untreated control group.

FIGS. 9A-D are photographs taken at 72 hours after treatment of HMS cells with the composition according to the invention (the experimental group).

FIGS. 10A-D are photographs taken at 72 hours after treatment of HMS cells without any agents (the untreated control group).

FIG. 11 is the autopsy photo, taken after the composition according to the invention was administered five times for 8 days from the ligation surgery in rats. Herein, the white arrow indicates the site of the ligation surgery, and the yellow arrows indicate the femoral arteries.

FIG. 12 is the autopsy photo, taken after no treatment was done for 8 days from the ligation surgery in rats.

FIG. 13 is the autopsy photo, taken after the composition according to the invention was administered eight times for 14 days from the ligation surgery in rats. Herein, the white arrow indicates the site of the ligation surgery, and the yellow arrows indicate the femoral arteries.

FIG. 14 is the autopsy photo, taken after no treatment was done for 14 days from the ligation surgery in rats. Herein, the white arrow indicates the site of the ligation surgery, and the yellow arrows indicate the femoral arteries.

FIGS. 15 to 17 show the photographs taken at 0, 11 and 15 days after the composition according to the invention was administered to 5-week-old female nude mice (BALB/c nu/nu), respectively.

FIG. 18 shows the photograph taken at 9 days after administration to other nude mouse, and

FIG. 19 shows the photograph taken at 15 days after administration to other nude mouse.

### EXAMPLES

The present invention is described in further detail in the following Examples which are not in any way intended to limit the scope of the invention as claimed. In addition, it will appear to those ordinarily skilled in the art that various modifications may be made to the disclosed embodiments, and that such modifications are intended to be within the scope of the present invention.

### Example 1

2-methyl-butyric acid and alanine in a weight ratio of 2:1 were reacted at 120° C. and 3.0 atm for 30 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7˜3.5 atm for 8 minutes. After that, sinigrin was added to the products obtained by said reaction, and was then reacted at 100° C. and 2.0 atm for 10 minutes. After that, valine was added to the products obtained by said reaction, and was then reacted at −10˜30° C. and 2˜7 atm for 10 minutes, and in succession was reacted at 200˜230° C. and 1 atm for 10 minutes. In all of the reaction steps, water was used as the solvent.

The final resultant products were purified by two different processes respectively.

As the first purifying process, the final resultant products were dissolved in water at 115˜125° C., and then filtered using the 25 mm nylon syringe filter with 0.2 μm pore size, purchased from VWR. Then, a filtration was successionally performed using the same filter at the temperature of 75˜85° C., 55˜65° C., 27˜33° C. and 2˜22° C., respectively. After that, the filtered solution was vacuum-dried to obtain solid compounds.

As another purifying process, the final resultant products were dissolved in water at 110˜130° C., and then filtered using the 25 mm nylon syringe filter with 0.2 μm pore size, purchased from VWR. Then, a filtration was successionally performed using the same filter at the temperature of 70˜90° C. and 23˜27° C., respectively. After that, the filtered solution was vacuum-dried to obtain solid compounds.

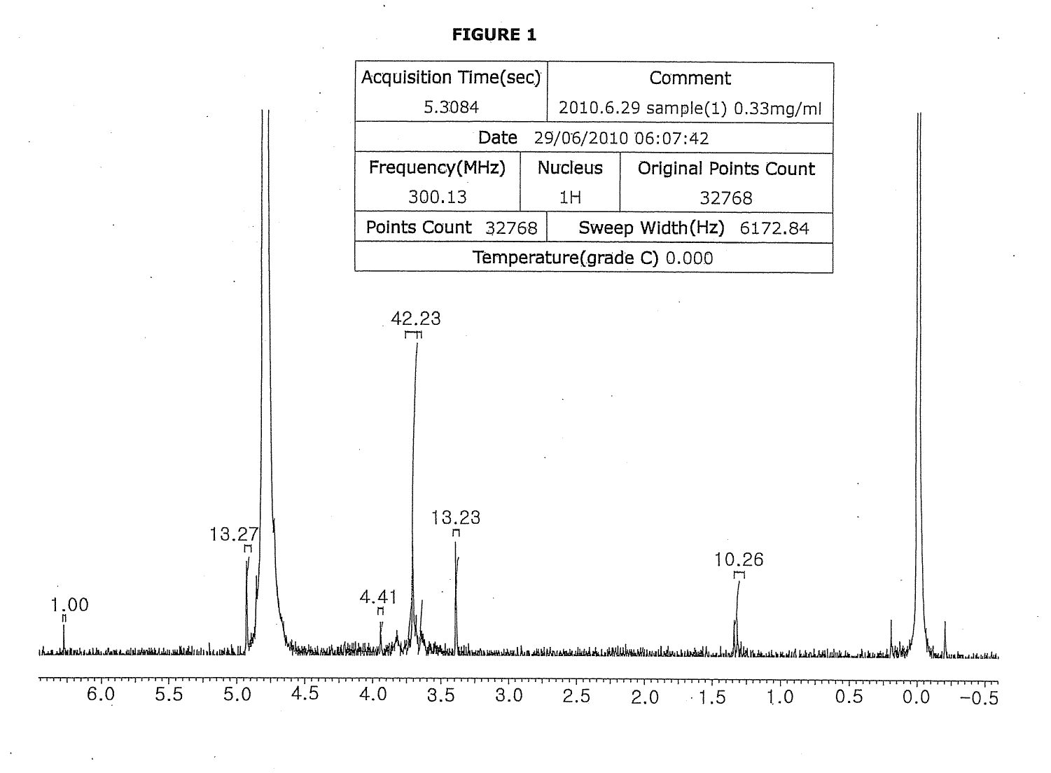

Both compounds obtained by said two processes show the ¹H NMR spectrum of FIG. 1, and are determined to have the following formula II:

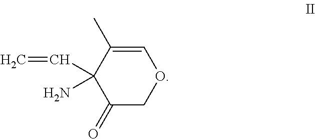

### Example 2

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7˜3.5 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1.

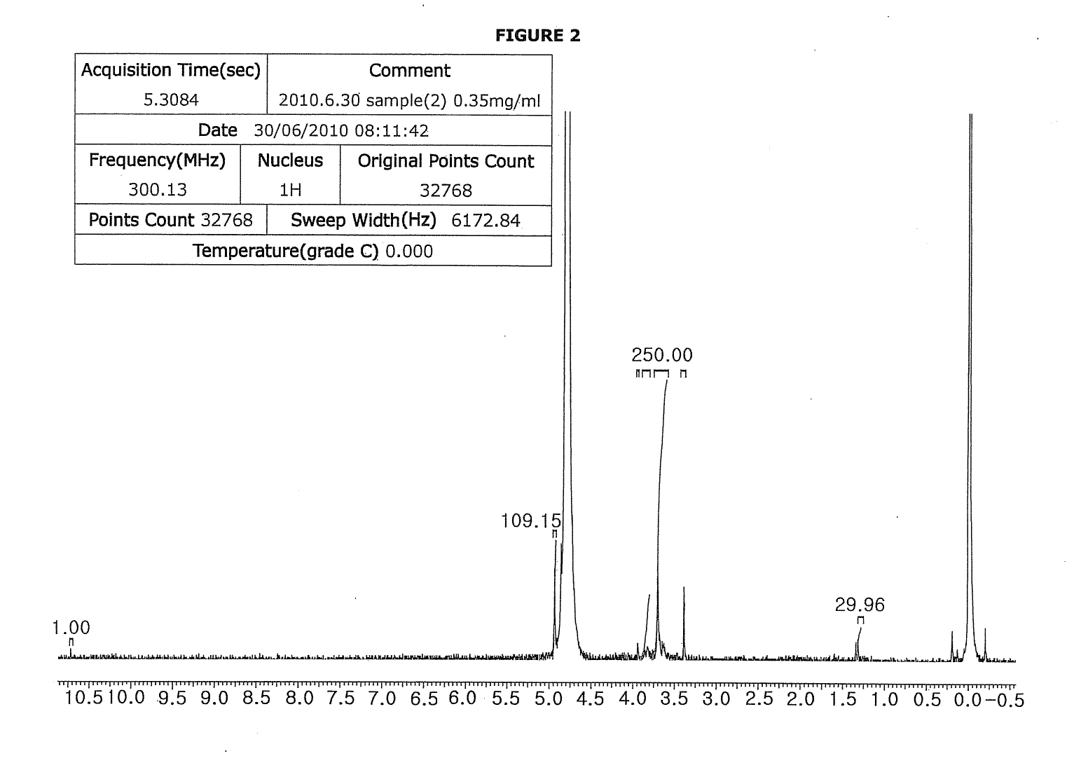

Both compounds obtained by said two processes show the ¹H NMR spectrum of FIG. 2, and are determined to have the following formula III:

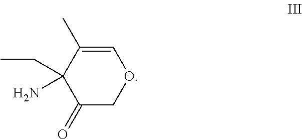

### Example 3

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and kaempferol in a weight ratio of 1:1 were added to the products obtained by said reaction, and were then reacted at 80˜120° C. and 1.3 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 4

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 5

2-methyl-butyric acid and arctigenin-4-O-glucoside in a weight ratio of 2:1 were reacted at 80˜120° C. and 2.7˜3.0 atm for 10 minutes. After that, copper and luteolin-7-rhamnoglucoside in a weight ratio of 3:1 were added to the products obtained by said reaction, and were then reacted at 120˜170° C. and 2.7 atm for 10 minutes. After that, vitexicarpin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 6

### Multiple Nuclear Divisions

Human Mammary Epithelial (HME) 50 HT cell line, which is the normal cell, was cultured in the Serum-free medium (SF-171 from Clonetics Corp., San Diego, Calif.).

Such a culture medium was treated with the composition comprising the compound of formula II obtained in the Example 1 and the compound of formula III obtained in the Examples 2 to 5, respectively.

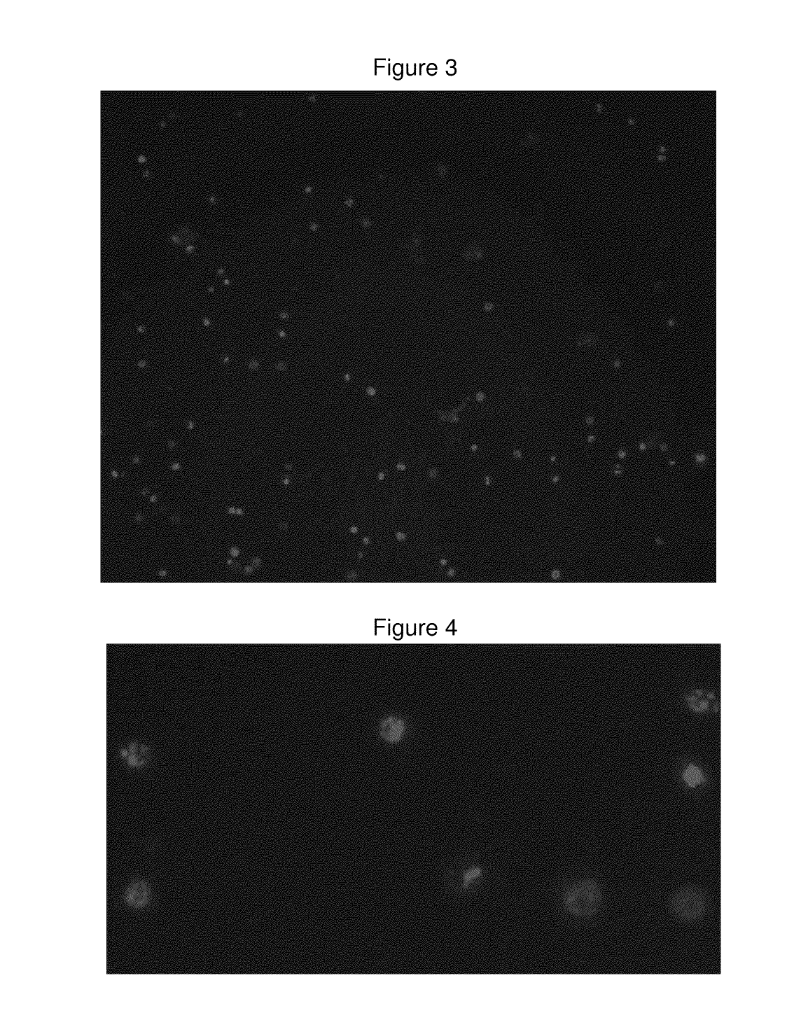

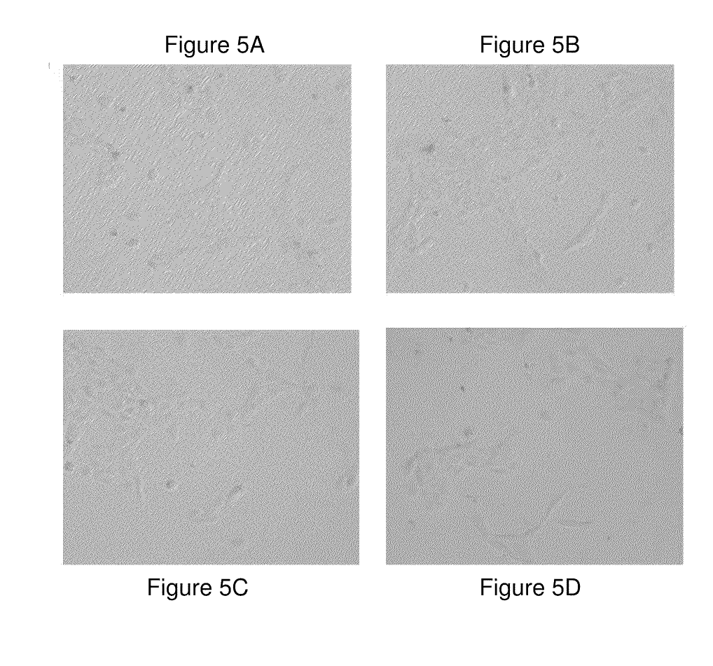

As a result, from the image of the stained-cell in FIG. 3 and the image magnified in FIG. 4, it was observed that multiple nuclear divisions occurred in the cell.

In addition, no cytotoxicity was observed in the group treated with the composition according to the invention. Surprisingly, the HME 50 HT cells even thrived and became over confluent during recovery. And, it was found from the photomicrographs that the cells appeared very healthy and unstressed.

### Example 7

### Regenerative effect on blood vessels and tissues

### In Vitro Experiments

HME 50 HT cells treated with the composition according to the invention in the Example 6 were continuously cultured. And, HMS cells were also treated and cultured in the same manner as the Example 6.

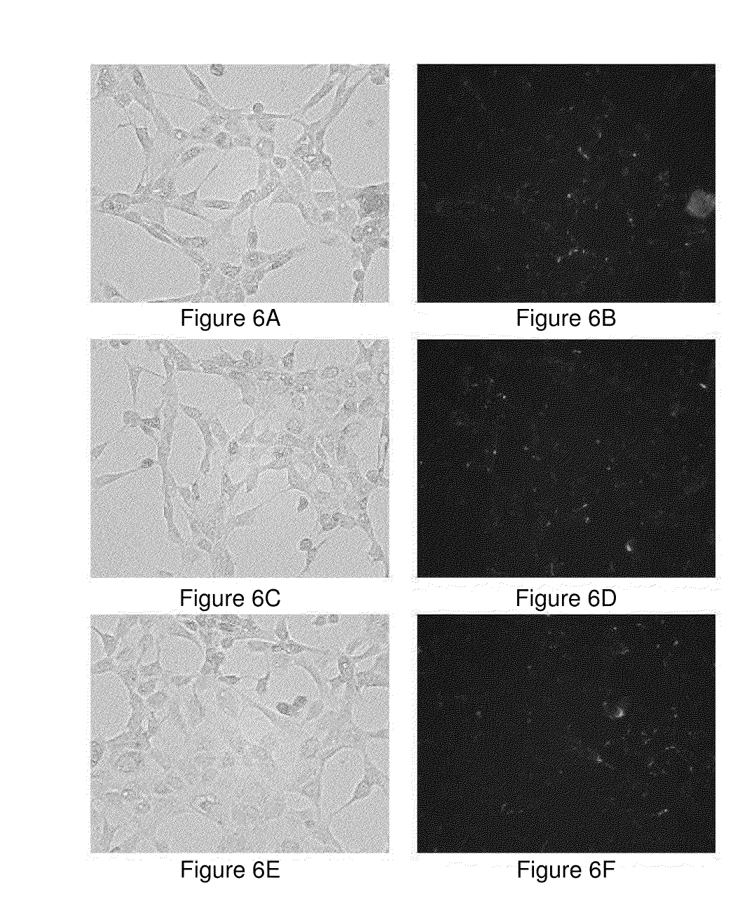

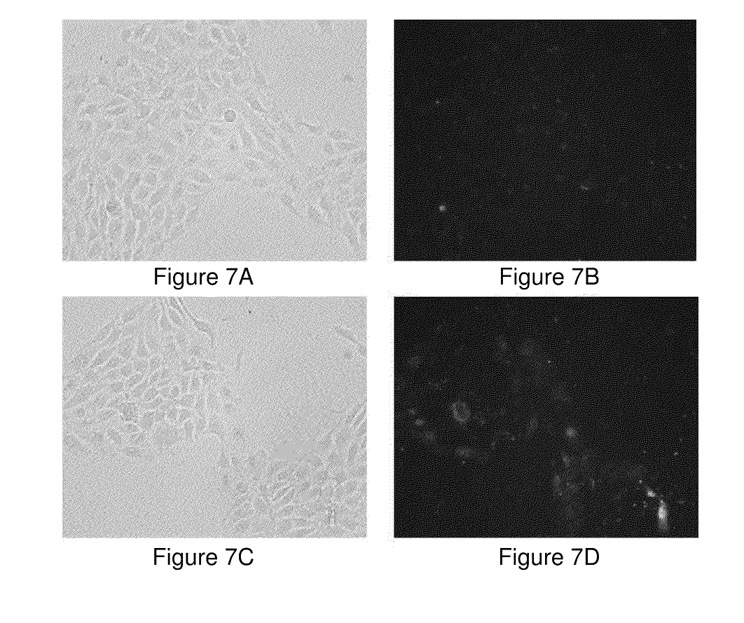

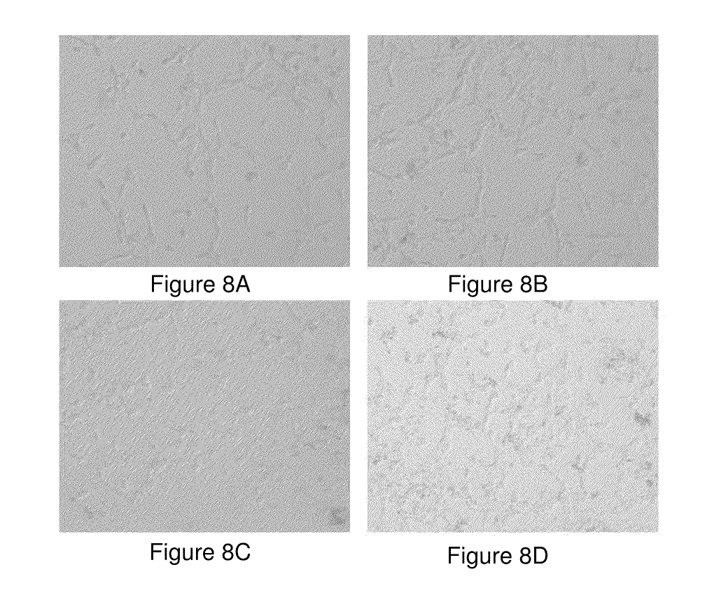

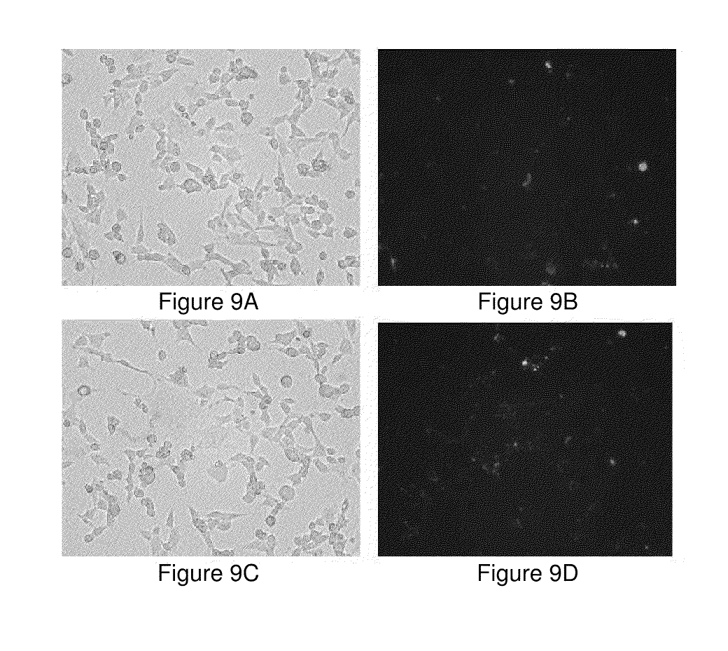

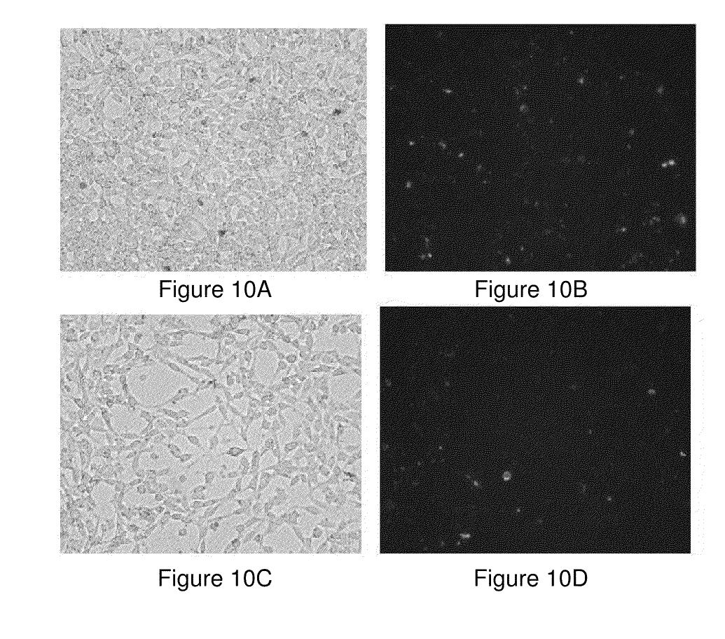

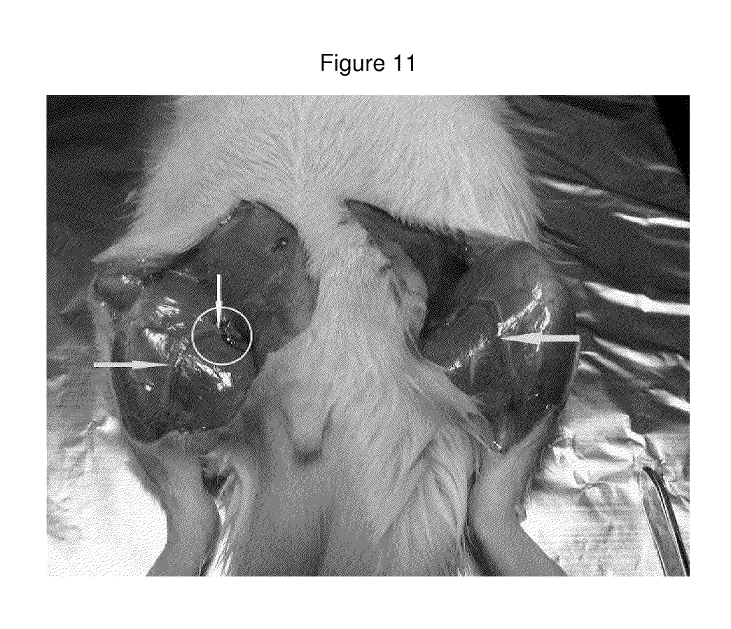

It was observed that the cells proliferated apart from each other and were randomly arranged in the untreated group. In contrast, in the experimental group treated with the composition according to the invention, cell association occurred and the cells were arranged at a regular pattern (FIGS. 5 to 10). This phenomenon means that the cells were in their early stages of making tissues.

(2) In Vivo Experiments

Ten Wistar rats were used as experimental animals. Hindlimb ischemic model was prepared by ligation of left femoral artery near the saphenous artery.

The composition according to the invention was orally administered to five rats at dose level of 0.33 mg/200 g (weight of compound/body weight of rat) at 9 a.m. daily during the experimental period (the experimental group), and any agents were not administered to other five mice (the control group).

3, 8 and 14 days after ligation of left femoral artery was undergone, respectively, they were sacrificed, and then autopsies were conducted. As samples, connective tissues and blood vessels except for muscles were taken at a distance of approximately 4 mm from the site of the ligation.

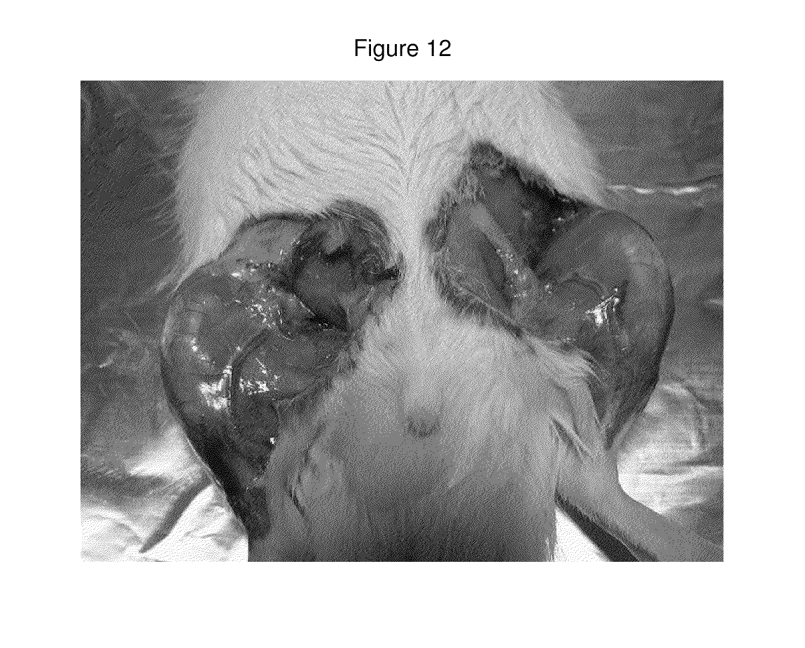

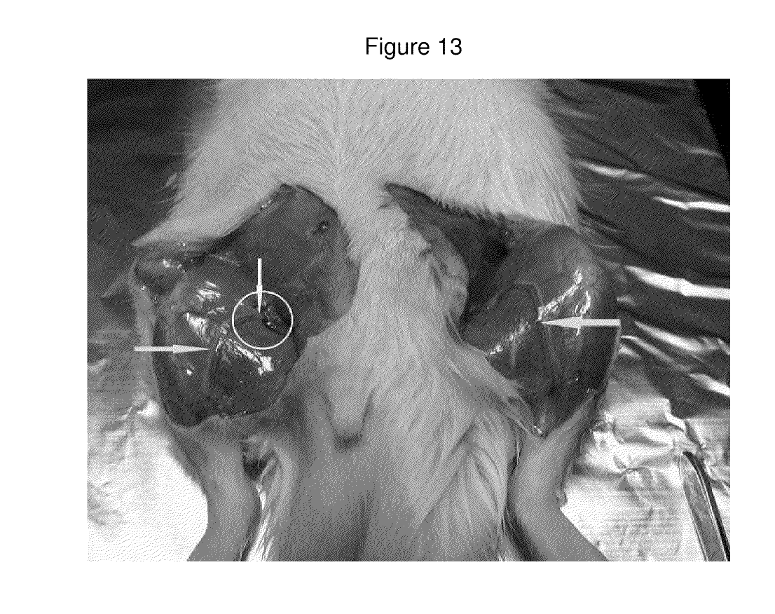

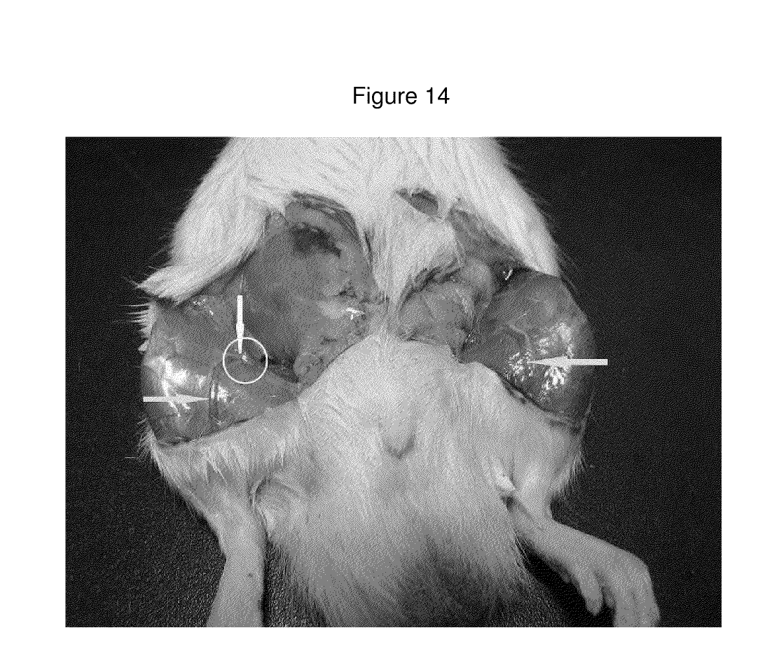

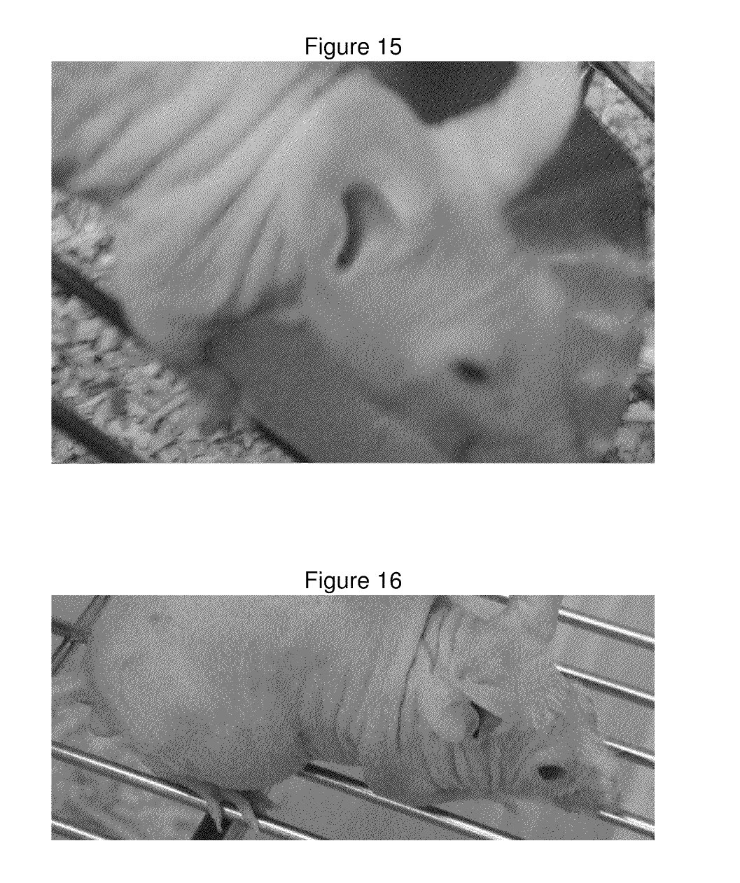

In the experimental group, several small-sized arteries were observed although large-sized arteries were not (FIGS. 11 and 13). Whereas, in the untreated control group, any bloodstream including small-sized arteries were not observed (FIGS. 12 and 14).

### Example 8

### Differentiation of T lymphocytes in Nude Mice

It is generally known in the art that the one of main defects of mice homozygous for the nude spontaneous mutation (Foxnl^{nu}) is defective development of the thymic epithelium. Nude mice are athymic caused by a developmental failure of the thymic anlage. Consequently, homozygous nude mice lack T cells and suffer from a lack of cell-mediated immunity. Because of a defect in helper T-cell activity, responses to thymus-dependent antigens when detectable are primarily limited to IgM. Homozygous nude mice show partial defect in B cell development probably due to absence of functional T cells. Other endocrine and neurological deficiencies have been reported.

PBS was orally administered to three 5-week-old female nude mice (BALB/c nu/nu), and the compound of the formula II according to the invention was orally administered to the other three 5-week-old female nude mice (BALB/c nu/nu) at dose level of 0.00033 mg/g (weight of compound/body weight of mouse) twice a day for 18 days.

FACS analysis was performed to identify the number of T lymphocytes in the Peripheral Blood Mononuclear Cells (PBMC) of nude mice. Anti-mouse CD8 was used as primary antibody, and FITC-conjugated rat anti-mouse IgG was used secondary antibody.

The result is shown in the Table 1. The number of T lymphocytes in the experimental group is twice as much as the number in the control group treated with PBS, and is close to the number in wild type rats.

TABLE 1

| CD8-positive T lymphocyte % | PBMC |
|---|---|
| Wild type rat | 4.92 |
| Nude mouse treated with PBS | 1.94 |
| Nude mouse treated with the composition | 3.84 |
| according to the invention | |

The result demonstrates that the composition according to the invention induced adult stem cells to differentiate into T lymphocytes, since the increase in T lymphocytes population can occur only through stem cell differentiation in nude mouse without thymus.

### Example 9

### Hairs in Nude Mice

The compound of formula II obtained in the Example 1 and the compound of formula III obtained in the Examples 2 to 5 were mixed with purified water, respectively. The prepared liquid-phase composition was at the concentration of 0.33 mg of compound per 1 ml of solution. In the following, the value for the composition according to the invention indicates the mean value of values obtained using the composition comprising the compound of formula II and of formula III.

It is generally known in the art that the one of main defects of mice homozygous for the nude spontaneous mutation (Foxnl^{nu}) is abnormal hair growth. Although the mice appear hairless, they are born with functional but faulty hair growth follicles. Hair growth cycles and patterns are evident especially in pigmented mice but the faulty follicles do not allow the hair to properly erupt.

During the experiment, the composition according to the invention was orally administered to 5-week-old female nude mice (BALB/c nu/nu) at dose level of 0.00033 mg/g (weight of compound/body weight of mouse) twice a day for 2 weeks. The group was consisted of 10 nude mice.

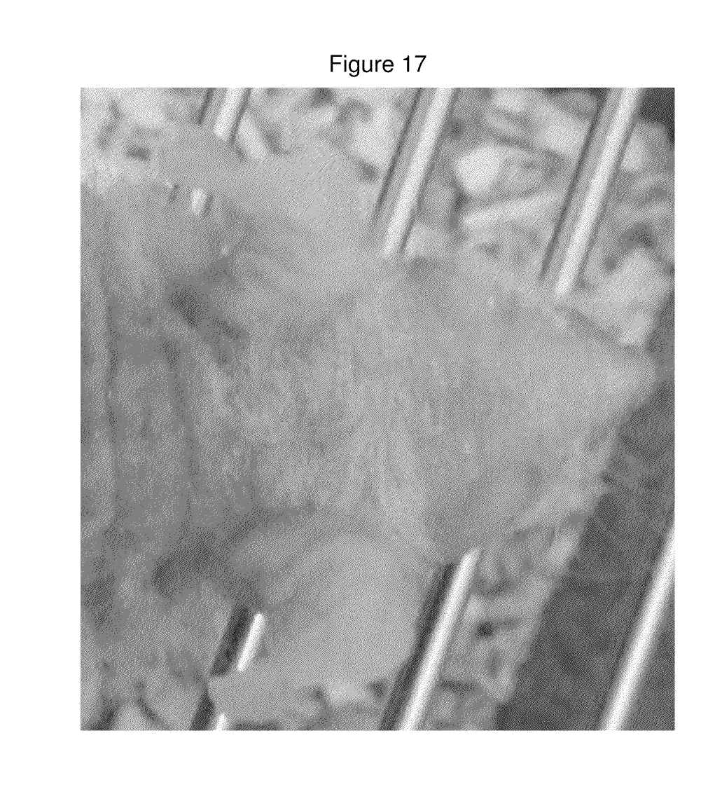

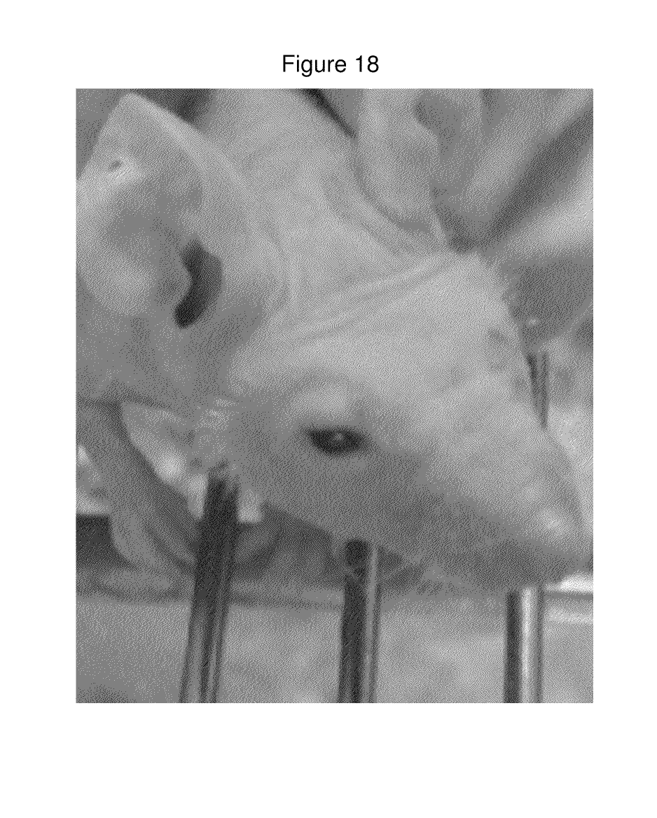

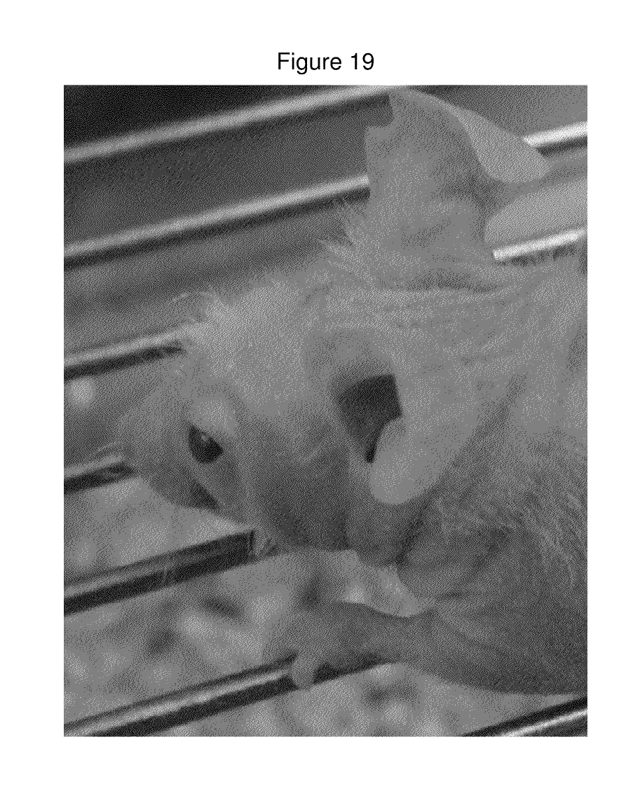

As a result, it is surprisingly found in all of the hairless nude mice that their hair started to grow on heads and flanks, as time goes by. In particular, at two weeks after administration of the composition according to the invention, it was observed that hair grew healthier and fuller in the nude mice. FIGS. 15 to 17 show the photographs taken at 0, 11 and 15 days after administration to no. 1 nude mouse, respectively. FIG. 18 shows the photograph taken at 9 days after administration to no. 2 nude mouse, and FIG. 19 shows the photograph taken at 15 days after administration to no. 7 nude mouse. This phenomenon means that abnormal genes in nude mice, after being treated with the composition according to the invention, changed into normal ones in wild type mice having white hair.

What is claimed is:

1. A composition for inducing multiple nuclear divisions of cells, comprising a compound of the formula I: 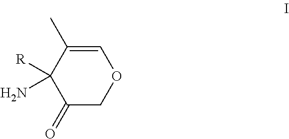
wherein R is C₂H₅ or C₂H₃,
or a pharmaceutically acceptable salt thereof.

2. The composition according to claim 1, wherein the composition induces tissue generation after the multiple nuclear divisions.

3. The composition according to claim 1, wherein the composition induces blood vessel generation after the multiple nuclear divisions.

4. The composition according to claim 1, wherein the composition induces organ generation after the multiple nuclear divisions.

5. The composition according to claim 1, wherein the composition induce stem cells to differentiate into immunocytes after the multiple nuclear divisions.

6. The composition according to claim 5, wherein the immunocytes are NK cell, NKT cell, T cell, B cell or thrombocytes.

7. The composition according to claim 1, wherein the composition is administered by parenteral, topical, oral, rectal or nasal route.

8. The composition according to claim 1, wherein the composition is used for treating or preventing cardiovascular diseases or immune deficiency.

9. The composition according to claim 2, wherein the composition is used for treating or preventing cardiovascular diseases or immune deficiency.

10. The composition according to claim 3, wherein the composition is used for treating or preventing cardiovascular diseases or immune deficiency.

11. The composition according to claim 4, wherein the composition is used for treating or preventing cardiovascular diseases or immune deficiency.

12. The composition according to claim 5, wherein the composition is used for treating or preventing cardiovascular diseases or immune deficiency.

13. A method for inducing multiple nuclear division of cells, comprising administering to the cells a compound of formula I 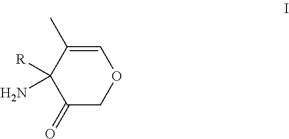
wherein R is C₂H₅ or C₂H₃,
or a pharmaceutically acceptable salt thereof.

14. A method for promoting the regeneration of blood cells or tissues in a human or animal subject, comprising administering to the subject a compound of formula I 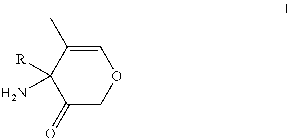
wherein R is C₂H₅ or C₂H₃,
or a pharmaceutically acceptable salt thereof.

15. A method for treating or preventing cardiovascular disease or immune deficiency according to claim 14.

16. The method of claim 14, wherein the compound is administered by parenteral, topical, oral, rectal or nasal route.
